(19) United States
(12) Patent Application Publication
(10) Pub. No.: US 2010/0167329 A1
(43) Pub. Date: Jul. 1, 2010

(54) DEVICE FOR DRUG SCREENING

(75) Inventor: Mats Wokander, Lund (SE)

Correspondence Address: MERCHANT & GOULD PC, P.O. BOX 2903, MINNEAPOLIS, MN, 55402-0903, US

(73) Assignee: Respiratorius AB, Lund (SE)

(21) Appl. No.: 12/444,553

(22) PCT Filed: Oct. 5, 2007
(86) PCT No.: PCT/SE2007/050718

§ 371 Date: Mar. 9, 2010

Related U.S. Application Data
(60) Provisional application No. 60/850,165, filed on Oct. 6, 2006.

(30) Foreign Application Priority Data
Oct. 6, 2006 (SE) 0602103-4

(51) Int. Cl. C12Q 1/02; C12M 1/34; G01F 23/292

(52) U.S. Cl. 435/29; 435/287.3; 250/577

(57) ABSTRACT

The present invention discloses a device and an optical liquid sensor for drug screening, and a method for using these. More particularly a device, an optical liquid sensor, and a method, for repeatable measurements of the effect of a test drug on a biological tissue sample are described.

### FIELD OF THE INVENTION

The invention relates generally to a device for drug screening. More particularly the invention relates to a test chamber for performing drug screening by a perfusing media, comprising a drug in a physiological saline solution. Even more particularly the invention relates to a liquid level sensor for controlling the level of the perfusing media.

### BACKGROUND OF THE INVENTION

Drug screening relates to the process of evaluating the effect on a sample of different candidate drugs in order to identify substances, which may be useful as pharmaceuticals. A typical basic method of drug screening involves contacting a sample with different substances and measuring the reaction of the sample to the different substances.

Screening of potential drug candidates presents many difficulties and challenges. It is desired to attain a high repeatability of the drug evaluations; a property which may be hard to obtain as fragile biological materials is often used.

When measuring contractions or relaxations in biological tissue the forces are very small. An inconsistent liquid level in the chamber will influence the measurements as the surface tension is significant. The liquid level in the chamber will vary due to evaporation of the fluid, temperature fluctuation and variation in the performance of the pumping system. There is thus a need for a device for conducting drug screening on smooth muscle tissue samples wherein the liquid level in the chamber is precisely maintained.

In the U.S. application US2005163712 a method for identifying a bronchi constriction relaxing substance is disclosed. Bronchial tissue samples in pairs are mounted to force transducers in a test chamber. By subjecting the tissue sample first to a contracting agent and then to a test drug, the contraction-relaxing effect of the test drug can be evaluated from the difference in tension in the tissue sample when subjected to the different substances. The test chamber is continuously perfused with physiological saline solution. The application does not disclose any device or any means for controlling the liquid level in the test chamber, thus this application is not applicable for repetitive experiments.

Liquid level sensors are known in the art. Examples of technologies which have been used are floats, radar or optical means.

U.S. application US20060117847 discloses a liquid level sensor, wherein a float is placed in a cylindrical tube which is placed in the fluid to be measured. Liquid is free to enter the tube and lifting the float. The position of the float is registered by infrared ray or supersonic means. A major drawback of this sensor is that the float and the tube disturb the surface of the liquid.

U.S. application US20050017895 describes a radar level measuring device for measuring a liquid level, wherein a radar transmitter is mounted above a light guide communicating with the fluid. Microwave radiation in a first and second plane of polarization is reflected at the liquid surface and at a reflector of known position respectively and the liquid level can be obtained. Drawbacks for this device include the light guide which is immersed and thus disturbs the surface of the liquid, and the radar which is not suitable for use in miniaturized equipment.

EP0274091 describes an optical displacement sensor. The device directs light to and from the liquid surface through fibre optics and the field of use is in measuring the liquid level in a tank. The sensor uses a reference beam. A main drawback of this sensor is that a reference measurement is needed. The sensor is thus not suitable for use in miniaturized equipment.

EP0601867 discloses a liquid supply control arrangement for controlling the supply of liquid to a container for applying a layer of molten hot melt to a shoe. EP0601867 only discloses the regulation of supply to a container.

WO 99/56093 discloses an optical sensor used for remote laser level monitoring in liquid storage vessels.

There is thus a need for an improved optical level sensor and a device comprising such a sensor for use in biological tissue measurements.

### SUMMARY OF THE INVENTION

Accordingly, the present invention seeks to mitigate, alleviate or eliminate one or more of the above-identified deficiencies and to provide an improved measuring device of the kind referred to, and a measuring method using said measuring device. For this purpose a device for performing drug testing and screening of substances having a contractile/relaxing effect on a tissue sample comprises

a chamber defining a volume, said chamber further comprising an inlet and outlet to said chamber, whereby a perfusion liquid, comprising said drug, may enter and leave said chamber, wherein said perfusion liquid transporting said drug to said biological tissue sample in said chamber,

an optical liquid level sensor for controlling the liquid level in said chamber,

mounting means for mounting said at least one tissue sample to force transducers, and

measuring means for measuring a contraction or relaxation of said at least one tissue sample by a signal from said force transducers.

Also, a liquid level sensor for measuring a distance to a surface to be measured, is provided, which optical liquid sensor comprises:

a light source,

a light transmitting optical fiber for transmitting light from said light source to the surface to be measured,

a light receiving optical fiber for receiving light reflected from the surface to be measured, and

a reflected light sensor for transducing a reflected light into a reflection signal.

Advantageous features of the invention are defined in the dependent claims.

### BRIEF DESCRIPTION OF THE DRAWINGS

These and other aspects, features and advantages of which the invention is capable of will be apparent and elucidated from the following description of embodiments of the present invention, reference being made to the accompanying drawings, in which

FIG. 1 is an illustration of a device according to one embodiment of the present invention,

FIG. 2 is a diagram showing long time stability of a tissue sample in a device according to one embodiment of the present invention, and

FIG. 3 shows contractions in two tissue samples in a device according to one embodiment of the present invention.

### DETAILED DESCRIPTION OF THE INVENTION

Chamber

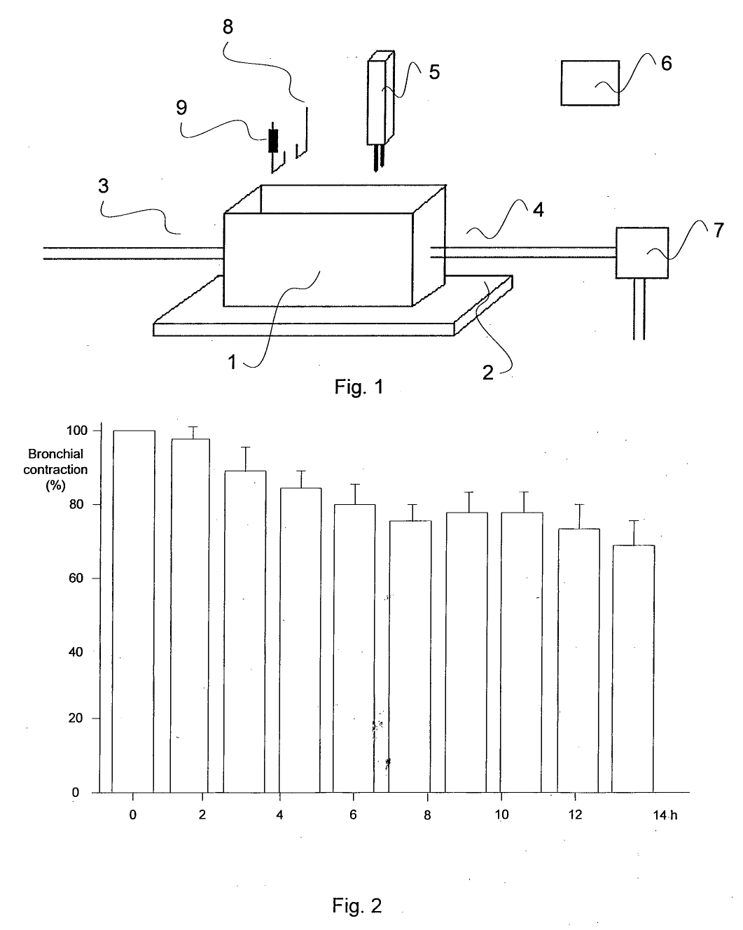

One embodiment of the present invention is illustrated in FIG. 1, wherein the chamber 1 is made out of titanium and has a volume of typically 5 mL. The chamber is mounted on a mounting piece 2 of silver, which contains means for controlling the temperature of the chamber. An inlet 3 and outlet 4 allow a continuous flow of a physiological solution through the chamber ensuring a constant concentration of the substances in the chamber. Tissue sample is placed on hooks 8 and the hooks are placed in the chamber in a suitable position so that the tissue sample is completely immersed in the liquid flowing through the chamber. One of the hooks is attached to a force transducer 9 and the other is a fixed hook. Test substances are injected in the liquid prior to the liquid entering the chamber. As the tissue sample reacts to the substances present in the liquid the force transducer register the tension in the tissue sample due to contraction or relaxation. The force transducer is preferably AME801, which is commercially available from Infineon technologies.

Thus, in one embodiment of the present invention a device is provided, which device, comprising a chamber having a wall and a bottom defining a chamber interior volume that will hold liquid and is open at the top. This chamber may be a thermo stated chamber defining a volume. This volume may be defined by a wall, such as in a cylindric chamber, or a plurality of walls, such as in a cubic chamber. Naturally, the chamber may have any other number of walls, such as 3 or 5, as long as a volume is defined that will hold liquid. In another embodiment of the present invention the chamber is provided with an opening at the top for performing drug testing and screening of substances having a contractile/relaxing effect on a tissue sample. The chamber may also be provided with an inlet and outlet to the chamber interior volume for continuous flow of perfusion liquid therethrough, which perfusion liquid may transport a test drug to the biological tissue sample.

In one embodiment the chamber is provided with a temperature sensor and heating and cooling means for maintaining a specified temperature in the chamber, an optical liquid level sensor and control means for automatically keeping a specified liquid level in the chamber, at least one pair of hooks for mounting at least one tissue sample to force transducers, means for measuring a contraction or relaxation of the tissue sample through measuring a signal from said force transducer, and/or means for viewing and storing data related to said signal.

According to another embodiment of the invention an optical sensor is disclosed wherein light from a light source is transmitted through a light transmitting optical fiber to the surface to be measured and reflected light is transmitted through a light receiving optical fiber to a reflected light sensor for transducing a reflected light into a reflection signal.

Optical Liquid Level Sensor

The liquid level in the chamber is of critical importance and is monitored by an optical liquid level sensor 5. Light from a light source in the optical liquid level sensor may be guided trough a light transmitting fiber to the liquid surface. Light reflected at the fluid surface may then be guided through a light receiving optical fiber to a reflected light sensor, transducing the reflected light into a reflected light signal. The sensor may be connected to a control circuit 6. The control circuit may receive a signal from the sensor and compare the signal with a set value. If the signal represents a non-acceptable liquid level then the control circuit may operate valve 7, which drains the chamber until the correct liquid level is reached. By means of the liquid level sensor 5 the liquid level may be maintained within 100 μm of the specified level. The optical liquid level sensor does not employ a reference measurement as the out-put of the light source is stable during the course of a run. Thus said optical liquid level sensor is simpler to manufacture and more suitable to miniaturized equipments such as the chamber of the present invention.

Reproducibility

A description of embodiments of the device according to the invention is shown in the previous section. An example of long time stability of a tissue sample and repeatability of measurements is shown in FIG. 2. In FIG. 2 control contractions in small human bronchi exposed to repeated (n=8) cycles with LTD4 (10 nM, 30 min) and LTD4 free solution (60 min) for 14 hours, are shown. The error bars show a low variance and good repeatability. Almost 80% of the original contraction of the bronchi remains after 14 h of measurements.

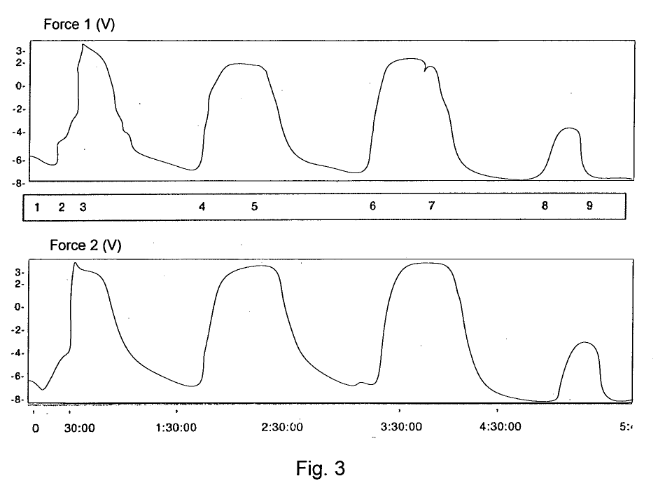

FIG. 3 shows an experiment where two tissue samples from the same source were tested simultaneously using different test drugs. Contractions in 2 tissue samples (upper and lower) mounted in the chamber upon simultaneous exposure to different substances during the course of a run are further shown in FIG. 3.

Thus, according to one embodiment of the present invention, a device, more specifically a thermo stated chamber having a wall or walls and a bottom defining a volume that will hold liquid and having an opening at the top for performing drug testing and screening of substances having a contractile/relaxing effect on a tissue sample comprising: a chamber having a wall and a bottom defining a chamber interior volume that will hold liquid and is open at the top, said chamber further comprising an inlet and outlet to the chamber interior volume for continuous flow of perfusion liquid therethrough, said perfusion liquid transporting a test drug to the biological tissue sample, a temperature sensor and heating and cooling means for maintaining a specified temperature in the chamber, an optical liquid level sensor and control means for automatically keeping a specified liquid level in the chamber, at least one pair of hooks for mounting at least one tissue sample to force transducers, means for measuring a contraction or relaxation of the tissue sample through measuring a signal from said force transducer, and means for viewing and storing data related to said signal, is provided.

According to another embodiment the device according to above is provided, wherein said test drug is injected into the perfusion liquid prior to the perfusion liquid entering the chamber.

According to still another embodiment the device according to above is provided, wherein said chamber has a rectangular horizontal cross section.

According to yet another embodiment the device according to above is provided, wherein said chamber has a volume of 0.1 mL to 20 mL and more preferably 1 mL to 5 mL.

According to another embodiment the device according to above is provided, wherein the chamber is made of titanium.

According to still another embodiment the device according to above is provided, wherein the chamber is mounted on a silver slab containing means for measuring temperature and controlling temperature.

According to yet another embodiment the device according to above is provided, wherein said at least one pair of hooks are mounted in a position 0 to 100 mm above the bottom of the chamber.

According to a further embodiment the device according to above is provided, wherein one in each pair of the at least one pair of hooks is connected to a force transducer and the other hook is a fixed hook.

According to still another embodiment the device according to above is provided, wherein said liquid level sensor and control means maintain the liquid level in the chamber within 100 μm of the specified level.

According to another embodiment the device according to above is provided, wherein said biological tissue sample is mammalian lung tissue.

According to another embodiment the device according to above is provided, wherein said biological tissue is small human bronchi.

Also, an optical liquid level sensor for measuring a distance to a surface to be measured, is provided, which optical liquid sensor comprises:

a light source,

a light transmitting optical fiber for transmitting light from said light source to the surface to be measured,

a light receiving optical fiber for receiving light reflected from the surface to be measured,

a reflected light sensor for transducing a reflected light into a reflection signal.

According to another embodiment the optical liquid sensor according to above is provided, further comprising control means receiving a signal from said optical liquid level sensor and operating a valve controlling the flow to and from the chamber.

According to another embodiment the optical liquid sensor according to above is provided, wherein said light transmitting optical fiber and light receiving optical fiber are omitted and said light source and reflected light sensor are placed adjacent to each other facing the liquid surface for measuring the liquid level without the need for optical fibers.

According to another embodiment of the invention an optical sensor is disclosed wherein light from a light source is transmitted through a light transmitting optical fiber to the surface to be measured and reflected light is transmitted through a light receiving optical fiber to a reflected light sensor for transducing a reflected light into a reflection signal.

While the present invention has been disclosed with reference to embodiments, numerous modifications, changes and alterations to the described embodiments are possible without departing from the spirit and scope of the present invention as defined in the claims. Accordingly, it is intended that the present invention not be limited to the described embodiments, but that it has the full scope defined by the language of the claims, and equivalents thereof.

1. A device for measuring the effect of a drug on at least one tissue sample, comprising:
a chamber defining a volume, said chamber further comprising an inlet and outlet to said chamber, whereby a perfusion liquid, comprising said drug, may enter and leave said chamber, wherein said perfusion liquid transporting said drug to said biological tissue sample in said chamber,
an optical liquid level sensor for controlling the liquid level in said chamber, mounting means for mounting said at least one tissue sample to force transducers, and
measuring means for measuring a contraction or relaxation of said at least one tissue sample by a signal from said force transducers.

2. The device according to claim 1, comprising means for viewing and storing data related to said signal.

3. The device according to claim 1, comprising heating and cooling means.

4. The device according to claim 3, comprising a temperature sensor, configured with said heating and cooling means to maintain a specified temperature in said chamber.

5. The device according to claim 1, wherein said drug is injected into the perfusion liquid prior to the perfusion liquid entering the chamber.

6. The device according to claim 1, wherein said chamber has a rectangular horizontal cross section.

7. The device according to claim 1, wherein said chamber has a volume of 0.1 mL to 20 mL, and more preferably 1 mL to 5 mL.

8. The device according to claim 1, wherein the chamber is made of titanium.

9. The device according to claim 1, wherein the chamber is mounted on a silver slab containing means for measuring temperature and controlling temperature.

10. The device according to claim 1, wherein said mounting means comprises at least one pair of hooks that are mounted in a position 0 to 100 mm above the bottom of the chamber.

11. The device according to claim 10, wherein one in each pair of the at least one pair of hooks is connected to a force transducer and the other hook is a fixed hook.

12. The device according to claim 1, wherein said liquid level sensor maintains the liquid level in the chamber within 100 μm of the specified level.

13. The device according to claim 1, wherein said biological tissue sample is mammalian lung tissue.

14. The device according to claim 1, wherein said biological tissue is small human bronchi.

15. An optical liquid level sensor unit for measuring a distance to a surface to be measured, which optical liquid sensor unit comprises:
an optical liquid level sensor, comprising
a light source,
a light transmitting optical fiber for transmitting light from said light source to the surface to be measured,
a light receiving optical fiber for receiving light reflected from the surface to be measured in a chamber, and
a reflected light sensor for transducing a reflected light into a reflection signal; and
control means for receiving a signal from said optical liquid level sensor and operating a valve controlling the flow from the chamber.

16. The optical liquid level sensor unit of claim 15, wherein said light transmitting optical fiber and light receiving optical fiber are omitted and said light source and reflected light sensor are placed adjacent to each other facing the liquid surface for measuring the liquid level without the need for optical fibers.

17. A method for measuring the effect of a drug on at least one tissue sample, comprising:
perfusing said at least one tissue sample with a perfusion liquid in a chamber defining a volume, said chamber further comprising an inlet and outlet to said chamber, whereby said perfusion liquid, comprising said drug, may enter and leave said chamber, wherein said perfusion liquid transporting said drug to said biological tissue sample in said chamber,
controlling the liquid level in said chamber with an optical liquid level sensor,
mounting said at least one tissue sample to force transducers, and
measuring a contraction or relaxation of said at least one tissue sample by a signal from said force transducers.
